(19) United States
(12) Patent Application Publication
(10) Pub. No.: US 2005/0042184 A1
(43) Pub. Date: Feb. 24, 2005

(54) Solid oral anti-tartar and anti-plaque compositions

(76) Inventors: Roberto Colle, Lainate (IT); Guglielmo Salmoiraghi, Lainate (IT); Andrea Barrica, Lainate (IT)

Correspondence Address: YOUNG & THOMPSON, 745 SOUTH 23RD STREET, 2ND FLOOR, ARLINGTON, VA, 22202, US

(21) Appl. No.: 10/494,570

(22) PCT Filed: Nov. 5, 2002
(86) PCT No.: PCT/EP02/12329

(30) Foreign Application Priority Data
Nov. 6, 2001 (IT) MI2001A002320

(51) Int. Cl. A61K 7/18; A61K 7/16

(52) U.S. Cl. 424/52; 424/56; 424/57

(57) ABSTRACT

Oral formulations in the form of chewing gum comprising: a. polyphosphates; b. hydrated silica; c. a source of fluoride ions; d. a polymer derived from chitin, or other naturally occurring hydrocolloids or a mixture thereof; e. optionally extracts or active ingredients of vegetable origin and/or antibacterial/disinfectant agents. The compositions of the invention are useful as adjuvants in dental hygiene, in particular to reduce tartar deposits

The present invention relates to oral anti-tartar and anti-plaque compositions, useful as adjuvants in odontostomatological hygiene.

### BACKGROUND TO THE INVENTION

The problem of dental plaque and tartar formation has long been studied, and agents which can be used to combat and delay such formation are being actively researched.

The mechanisms that cause tartar deposits are well known; these deposits are constituted by calcium phosphate crystals which precipitate in the extracellular matrix of bacterial plaque. The pathogenetic role of tartar in periodontal diseases such as pyorrhoea, periodontitis, gingivitis and correlated disorders is equally well known.

Various substances have proved effective in reducing or preventing tartar formation and deposits on the teeth, including soluble pyrophosphates and polyphosphates, zinc salts, fluorides, diphosphonates, antibacterial agents such as triclosan, and abrasive agents such as silica or alumina. These substances, combined with one another in various ways, are included in the composition of most anti-tartar toothpastes now commercially available. The clinical efficacy of these toothpastes has been examined in numerous studies, reviewed in J. Clin. Dent. Vol IV(3), 71-81, 1993.

The most common toothpastes contain soluble polyphosphates associated with fluorides and silica, and possibly with polymers that possess bioadhesive properties, as described, for example, in U.S. Pat. No. 4,327,977, U.S. Pat. No. 4,889,713, U.S. Pat. No. 5,017,362, U.S. Pat. No. 5,139,769, U.S. Pat. No. 4,921,693 and EP 492997.

Similar compositions, with the addition of antibacterial agents such as triclosan, are described, for example, in GB 2200551. In addition to toothpastes, chewing gums and candies with a similar composition have been developed.

The efficacy of these toothpastes, which has been the object of numerous studies (J. Clin. Dent. Vol. X(3), 99-102, 1999; Oral Surg. Oral Med. Oral Pathol., Vol. 70(4), 529-536, 1990; J. Clin. Dent. Vol. IX(4), 101-104, 1998), is due to inhibition of calcium phosphate precipitation by the polyphosphates that complex the calcium ions in the saliva, to the abrasive action of silica, to the reinforcing effect of fluorides on the tooth enamel and to the action of bioadhesive polymers, where used, which protects the mucosae and causes slow release of the other ingredients.

The polymers most often used in compositions designed to control tartar are polycarboxylates derived from acrylic or methacrylic acid, particularly copolymers of maleic anhydride with methyl vinyl ether (GANTREX®). However, these polymers are not approved for use in foodstuffs, which means that they can only be used to make toothpastes and mouthwashes.

On the other hand, candies and chewing gum designed as adjuvants in dental hygiene and oral hygiene in general, which have properties that can be described as anti-tartar, anti-decay, whitening and/or refreshing, are becoming increasingly popular. The main advantage of these forms of administration is that they can be used freely and conveniently during the day in any place and on any occasion, in addition to that fact that the release of the active elements (functional ingredients) is slower and more regular than in the case of an ordinary toothpaste.

### DESCRIPTION OF THE INVENTION

The present invention relates to oral formulations in a solid form, preferably in the form of chewing gum, whose efficacy is superior to that of similar known formulations.

The compositions of the invention contain effective amounts of:
a. polyphosphates, preferably a mixture of alkali metal pyrophosphates and triphosphates;
b. an abrasive agent (preferably hydrated silica);
c. a source of fluoride ions;
d. a polymer derived from chitin, or other naturally occurring hydrocolloids or a mixture thereof;
e. optionally extracts or active ingredients of vegetable origin;
f. optionally antibacterial or disinfectant agents.

In addition to the active ingredients referred to above, the compositions of the invention will contain excipients suitable to define the final form of administration.

Thus, for example, a chewing gum formulation will require a suitable base consisting of gum base, sweeteners, polyalcohols such as xylitol, sorbitol and mannitol, flavourings, dyes, softeners, plasticisers, stabilisers, thickeners, etc.

The fact that the compositions of the invention comprise a system able to form a film on the oral mucosa increases protection against tartar deposits, because the active ingredients remain in contact with the user's teeth and gums for a longer time and the polyphosphates are protected against the hydrolysing action of the oral cavity.

In the present invention, this system consists of a chitin deacetylated derivative, possibly chemically modified and optionally in association with other polymers, to enhance its bioadhesive properties and its ability to protect the polyphosphates against hydrolysing agents.

There has been great scientific interest in controlled-release systems directed at the oral mucosa in the past decade (J. Clin. Phar. Ther. (2000) 25, 21-42). The polymers studied include partly deacetylated chitin, highly deacetylated chitin or chitosan and hydrolysed chitosan or oligosaccharide, which have proved able to adhere to the tissues thanks to the positive charges of the ammonium groups. Partly as a result of their bioadhesive properties, these polymers accelerate wound healing and haemostasis (Biom. 1999, 20(22): 2139-45; J. Oral. Max. Surg. 57: 49-52). Specific studies demonstrate the bioadhesive properties of chitosan towards the oral mucosa (Biom. 16 (1995) 617-624; J. Control Rel. 61: 175-183, Int. J. Pharm. 73: 43-48).

Among the forms described, the preferred form is chitosan oligosaccharide, a commercially available compound that comprises two to seven monomer D-glucosamine units bonded to one another with β-1,4 bonds, mainly obtained by enzymatic hydrolysis of chitosan with a higher molecular weight.

Although this procedure is known, it does not appear to have been applied in compositions similar to those which are the object of this invention.

Optionally, naturally occurring polysaccharides hydrocolloids may be used as alternative with similar bioadhesive properties. Polysaccharides hydrocolloids of this type are: xanthan gum, locust bean gum, alginates, carrageneen, gallan gum and others.

Broadly, the polymer-based system described above amounts to between 0.5 and 5% by weight on the total composition, and preferably between 1 and 3%.

The polyphosphates used in the compositions may be alkali metal pyrophosphates (diphosphates), hexametaphosphates, tripolyphosphates or mixtures thereof. A mixture of disodium diacid diphosphate and pentasodium or pentapotassium triphosphate is particularly preferred. It has been proved that a toothpaste containing this mixture provides a more marked reduction of tartar than a toothpaste containing pyrophosphates not associated with triphosphates (J. Clin. Dent. Vol. IX(4), 101-104, 1998).

Broadly, the polyphosphates amount to between 0.5 and 5% by weight on the total composition to which the invention relates.

The function of the abrasive agent is to increase the plaque-removing action already possessed by ordinary chewing gum. It may be formed by hydrated silica (in a suitable form), calcium carbonate (in a suitable form) or talc, either individually or combined with one another. These abrasives may also be present totally or partly, either individually or in a mixture thereof, in encapsulated form, in particular encapsulated in calcium alginate. Chewing gum containing microgranules of hydrated silica encapsulated in calcium alginate has proved more effective in removing plaque than a chewing gum with the same formulation but without microgranules (Doc. Os 06.2001 779-781). According to the present invention, the encapsulated microgranules may contain dyes, flavourings, functional ingredients and herb extracts. This abrasive agent is usually present in percentages of between 0.5 and 7% by weight.

Suitable sources of fluoride ions include sodium fluoride, potassium fluoride, ammonium fluoride, sodium monofluoro-phosphate and other known non-toxic salts containing fluorine, in concentrations which provide fluoride percentages of between 0.005 and 0.2% by weight.

The vegetable extracts which may be present in the compositions of the invention will preferably be selected from extracts of Centella asiatica, Malva sylvestris, Melaleuca alternifolia, Commiphora abyssinica (myrrh), Krameria triandi (rhatany), Acacia catechu, Medicago sativa (alfalfa), resins of the genus Styrax, such as Styrax benzoin (benzoin), Matricaria recutita (camomile), Echinacea purpurea (echinacea) and Croton lechleri (dragon's blood). Extracts of these plants, whose activity has been known for some time, are commercially available.

The combination with these extracts gives the formulations anti-inflammatory/decongestant, emollient, wound-healing, antiseptic and astringent properties. These properties are desirable in at least two respects in the ambit of the present invention:

firstly, to assist and reinforce the reduction in diseases of the oral mucosa caused by the reduction in tartar, and

secondly, to control and prevent contact stomatitis similar to that manifested with the use of toothpastes, known as “toothpaste stomatitis”, in particularly predisposed persons.

In the formulations of the invention, these extracts may be encapsulated in alginate together with the abrasive agent.

Said extracts may be added to the formulations in percentages of between 0.01 and 2% by weight.

The formulations of the invention can be prepared by conventional techniques, by adding and mixing the various ingredients to the gum base in the case of chewing gum, which may then undergo coating operations in accordance with equally conventional techniques.

The formulations of the invention may include disinfectant or antibacterial agents such as triclosan, zinc salts or zinc oxide, either alone or combined with one another, in concentrations of between 0.1 and 5% by weight. These agents are designed to combat the formation of bacterial plaque, which leads to tartar deposits.

The formulations of the invention may also include decorative crystals, preferably consisting of gum arabic and dyes deposited on the surface of the product with a purely aesthetic function.

Daily use of the chewing gum in accordance with the invention reduces tartar deposits and has other beneficial effects on the condition of the oral and gingival mucosa.

The following examples illustrate the invention in greater detail.

### EXAMPLE 1

### Coated Chewing Gum Weighing 1.4 g

Percentage composition (by weight)

| Ingredient | % |
|---|---|
| Gum base | 24.5 |
| Xylitol | 23.5 |
| Sorbitol | 23.2 |
| Mannitol | 16 |
| Flavouring | 1.8 |
| Silicon dioxide | 3 |
| Gum arabic | 1 |
| Glycerin | 1 |
| Disodium diacid diphosphate | 1 |
| Pentasodium triphosphate | 1 |
| Chitosan oligosaccharide | 1 |
| Maltitol syrup | 0.93 |
| Titanium dioxide (E171) | 0.7 |
| Quick Coat | 0.6 |
| Aspartame | 0.6 |
| Decorative crystals | 0.05 |
| Acesulfame | 0.05 |
| Carnauba wax | 0.05 |
| Potassium fluoride | 0.02 |
| | 100 |

### EXAMPLE 2

### Coated Chewing Gum Weighing 1.4 g with Vegetable Extracts

Percentage composition (by weight)

| Ingredient | % |
|---|---|
| Gum base | 24.5 |
| Xylitol | 23.5 |
| Sorbitol | 23.2 |
| Mannitol | 16 |
| Flavouring (*) | 1.8 |
| Silicon dioxide | 3 |
| Gum arabic | 1 |
| Glycerin | 1 |
| Disodium diacid diphosphate | 1 |
| Pentasodium triphosphate | 1 |
| Chitosan oligosaccharide | 1 |
| Maltitol syrup | 0.93 |
| Titanium dioxide (E171) | 0.7 |
| Quick Coat | 0.6 |
| Aspartame | 0.6 |
| Acesulfame | 0.05 |
| Carnauba wax | 0.05 |
| Potassium fluoride | 0.02 |
| Mallow, myrrh, centella, melaleuca, rhatany and | 0.05 |
| acacia catechu extracts. | |
| Total | 100 |

### EXAMPLE 3

### Efficacy Tests: Reduction in Tartar Deposit

A double-blind crossover clinical trial has been conducted to compare the effects of a chewing gum in accordance with Example 1 with those of a placebo gum.

28 Adults were admitted to the trial and treated with two chewing gums for five minutes, four times a day, for 6 weeks. At the end of this period a quantitative evaluation of the tartar deposit was carried out in accordance with the modified Volpe and Manhold index (J. Periodont. Res. (Suppl.) 14:31-60, 1974). Throughout the treatment period, the patients all used the same toothpaste (not containing anti-tartar agents) and followed a similar diet. The same patients were then treated for six weeks immediately after the first evaluation of the tartar deposit with the other chewing gum (Example 1 or placebo) in accordance with the same treatment procedure as before. At the end of the treatment period a second quantitative evaluation of the tartar deposit was carried out in accordance with the same procedure as described above.

The results were subjected to statistical analysis using Student's two-tailed paired sample “t” test.

The evaluation conducted after the patients had chewed the gum described in Example 1 demonstrated a 13.9% reduction in tartar deposits compared with those observed after chewing of the placebo gum. This reduction is statistically significant. The results of the study are summarised in Table 1.

TABLE 1
“T” test: paired samples for means

| | Placebo | Ex. 1 |
|---|---|---|
| Mean | 4.2410714 | 3.6517857 |
| Variance | 10.539269 | 7.9599041 |
| Observations | 28 | 28 |
| Pearson's correlation | | 0.9858011 |
| Hypothesised difference of means | | 0 |
| P(T <= t) two-tailed | | 6.884-05 |

1. Oral formulations in solid form, comprising:
a. polyphosphates;
b. an abrasive agent;
c. a source of fluoride ions;
d. a polymer derived from chitin, or other naturally occurring hydrocolloids or a mixture thereof;
e. optionally extracts or active ingredients of vegetable origin;
f. optionally antibacterial/disinfectant agents.

2. Formulations as claimed in claim 1, wherein the polyphosphates are selected from tripolyphosphates, pyrophosphates or mixtures thereof.

3. Formulations as claimed in claim 2, comprising a mixture of alkali metal pyrophosphates and tripolyphosphates.

4. Formulations as claimed in claim 1, further containing excipients selected from gum base, sweeteners, polyalcohols, flavourings, dyes, softeners, plasticisers, stabilisers and thickeners.

5. Formulations as claimed in claim 1, wherein the abrasive agent is hydrated silica, calcium carbonate and talc, either individually or in a mixture thereof.

6. Formulations as claimed in claim 1, wherein the chitin-derived polymer is a (partly or totally) deacetylated derivative of chitin, optionally chemically modified, optionally in association with other polysaccharides hydrocolloids.

7. Formulations as claimed in claim 6, wherein the polymer is a chitosan.

8. Formulations as claimed in claim 7, wherein the chitosan is chitosan oligosaccharide.

9. Formulations as claimed in claim 1, wherein the naturally occurring hydrocolloids are xanthan gum, locust bean gum, alginates, carrageeneens, gellan gum or other polysaccharides with bioadhesive properties.

10. Formulations as claimed in claim 1 containing extracts with anti-inflammatory, wound-healing, antihemorrhagic, soothing, emollient, decongestant and antiseptic properties.

11. Formulations as claimed in claim 10, wherein the vegetable extracts are selected from extracts of Centella asiatica, Malva sylvestris, Melaleuca alternifolia, Commiphora abyssinica (myrrh), Krameria triandra (rhatany), Acacia catechu, Medicago sativa (alfalfa), resins of the genus Styrax, such as Styrax benzoin (benzoin), Matricaria recutita (camomile), Echinacea purpura (echinacea) and Croton lechleri (dragon's blood).

12. Formulations as claimed in claim 1, containing 0.5 to 5% by weight of polyphosphates, 0.5 to 7% by weight of an abrasive agent (possibly wholly or partly encapsulated), 0.5 to 5% by weight of a polymer derived from chitin, and a source of fluoride ions able to guarantee a fluoride intake of 0.005 to 0.2%.

13. Formulations as claimed in claim 12, further containing 0.01 to 2% by weight of vegetable extracts.

14. Formulations as claimed in claim 1, comprising disinfectant and/or antibacterial agents selected from triclosan, zinc oxide and zinc salts, either alone or in combination with one another, in concentrations of between 0.1% and 5%.

15. Formulations as claimed in claim 1, wherein said formulations are chewing gum or candies.
